(19) United States
(12) Patent Application Publication
(10) Pub. No.: US 2024/0168113 A1
(43) Pub. Date: May 23, 2024

(54) RF PULSE GENERATION FOR MULTI-BAND MAGNETIC RESONANCE IMAGING

(71) Applicant: Resonance Research, Inc., Billerica, MA (US)

(72) Inventor: Hoby P. Hetherington, Columbia, MO (US)

(73) Assignee: Resonance Research, Inc., Billerica, MA (US)

(21) Appl. No.: 18/282,149

(22) PCT Filed: Mar. 18, 2022
(86) PCT No.: PCT/US2022/020928

§ 371 Date: Sep. 14, 2023

Related U.S. Application Data
(60) Provisional application No. 63/163,300, filed on Mar. 19, 2021.

(51) Int. Cl. G01R 33/36

(52) U.S. Cl. CPC G01R 33/3607

(57) ABSTRACT

A system and method of generating Radio Frequency (RF) pulses RF(t) in a Multi-band (MB) Magnetic Resonance Imaging (MRI) system is presented. The method includes determining a dirac comb function, the period of the comb function determining the frequency separation of the excitation bands. The method further includes determining a single band frequency selective pulse, defining the envelope of a pulse of RF(t), and the shape of individual excitation frequency bands in the frequency domain. The method further includes determining the shape of RF(t) during each period, and is used to modulate uniformity and/or number of individual excitation bands in the frequency domain. RF(t) pulses are generated in the MRI system simultaneously with an MRI gradient so as to simultaneously excite multiple slices within a subject, wherein the RF(t) pulse create a spin response.

### CROSS-REFERENCE TO RELATED APPLICATIONS

This application claims priority from U.S. Provisional Patent Application 63/163,300, filed Mar. 19, 2021, which is hereby incorporated herein by reference in its entirety.

### TECHNICAL FIELD

The present invention relates to Magnetic Resonance Imaging (MRI), and more particularly, to a system and methodology of RF pulse generation for Multi-band MRI.

### BACKGROUND ART

Magnetic Resonance Imaging (MRI) is a medical imaging technique used to form images of anatomy within the body. To generate these images, MRI systems use strong magnetic fields, magnetic field gradients, and radio waves. FIG. 1A-1C show a conventional Magnetic Resonance Imaging (MRI) system. As seen therein, the superconducting magnet system 10 includes a magnet housing 12, a superconducting magnet 13 with solenoidal windings whose axis of symmetry by convention is the Z-axis of a Cartesian coordinate system, shim coils 14, gradient coils 16, RF coils 18, and a patient table 20. As is well known in the art, the superconducting magnet 13 produces a substantially uniform magnetic B₀ field within its design FOV. This B₀ field is directed along the positive Z-axis. Such systems are useful for performing magnetic resonance investigations and are suitable for producing diagnostic images for human studies; similar systems can be used for spectral analysis applications.

As shown in FIG. 1B, the targeted subject 25 is to be placed in the middle of the main magnetic field B₀ generated by the superconducting magnet 13. The voltage measurements are registered by measurement circuitry 40. Voltage measurements are used to reconstruct images and the images are displayed on a controller/display 50. Preferably, the application of current is synchronized with the magnetic resonance imaging sequence from a magnetic resonance imaging system 60. A greater detailing of functions is set for by Prior Art FIG. 1C.

Thus, the basic hardware components of all high field strength MR systems conventionally are: the superconducting magnet which produces a stable and very intense magnetic field; the gradient coils which create a variable field to enable spatial encoding; and the radio frequency (RF) coils which are used to stimulate transitions between the energy states of the nuclei. A controller which may include a computer with software controls the scanning procedure and processes the information.

More particularly, MRI systems typically employ a spatially uniform and temporally constant main B₀ magnetic field. For the purpose of excitation of nuclear spin magnetization within the examination volume of the magnetic resonance device, a radio frequency (RF) pulse sequence, the B₁ field, is superimposed perpendicular to the B₀ field at the appropriate resonant frequency.

Conventional magnetic resonance imaging devices typically include a set three of gradient coils for the generation of the linear gradient magnetic fields, by which spatial encoding of the nuclear spin magnetization is achieved. During the magnetic resonance procedure, pulse sequences (consisting of radio frequency and switched gradient magnetic fields) are applied to a targeted subject (such as a live patient) to generate magnetic resonance signals, which are detected and stored to obtain information subsequently used to reconstruct spectra and images of the object. These procedures determine the characteristics of the reconstructed spectra and images such as location and orientation in the targeted subject, dimensions, resolution and contrast. The operator of the magnetic resonance device typically selects the appropriate sequence and adjusts and optimizes its parameters for the particular application.

Illustratively in multi-slice 2D imaging, a slice gradient is provided along an axis perpendicular to the plane of the desired slice, causing potential resonance frequencies in that direction. An RF pulse is simultaneously applied whose narrow frequency matches that of the desired slice, such that only protons within the desired slice are excited.

Multi-band (MB) Imaging, also known as Simultaneous Multi-slice (SMS) imaging, increases the speed for multi-slice 2D imaging by acquiring multiple slices simultaneously. See, for example, Larkman, D. J., et al., Use of multicoil arrays for separation of signal from multiple slices simultaneously excited. J Magn Reson Imaging, 2001. 13(2): p. 313-7, which is hereby incorporated by reference herein it its entirety. To achieve this, multiple spatially separated slices are excited simultaneously by applying a specialized RF pulse during a slice selective gradient (FIG. 2A) to simultaneously excite multiple slices (FIGS. 2B and 2C (three slices)) and the resulting overlapping data are resolved using multi-channel receive arrays. The varying spatial dependence of the individual receive coils enables data from the simultaneously excited slices to be separated into their original slices, as shown in FIG. 3. Thus two enabling components of MB or SMS are: 1) RF waveforms which simultaneously excite multiple slices and 2) data reconstruction methods that utilize the spatial dependence of multiple receive coils to resolve the overlapping data.

Conventional Multiband RF Excitation: Multi-slice excitation is typically achieved using a complex summation of RF waveforms designed to excite the individual single slices. The complex summation assumes a single base frequency for one rf pulse. For example, the rf pulse may be a pulse defined by equation 1. Additional pulses are added, modulating their phase according to the difference between their frequency and that of the reference pulse, as defined by equation 2 and shown in FIG. 4A. In the example shown in FIG. 4b, three distinct slices are excited with a single complex waveform with SB=[−1,0,+1]. The frequency selectivity of each excitation band in FIG. 4B is maintained, as shown in FIG. 4C.

RF(t)=RF₀(t)*e^{-iPhase}^{₀}(t) Eq. 1

RF(t)=Σ_{SB}RF₀(t)*e^{-i(Phase}^{₀}^{(t)+SB*ωt)}SB=[−n . . . +n] Eq. 2

The complex summation results in a rapid oscillation of the amplitude, as shown in FIG. 4A, which increases in relative amplitude (3 fold) of the applied RF when the phases are constructive and decreases when the phases are destructive. For high multi-band factors the peak amplitude of the combined RF waveforms can become large and result in substantial increases in deposited power. Notably the peak amplitude of the multi-band pulse increases by a factor of 3, or 9 in power in comparison to the conventional single band pulse. The overall power deposition increases by a factor of 5.

Multiband Excitation by Interdigitating Pulses and Delays: Alternatively, multiple excitation bands can be created by interdigitating RF pulses with periods where no RF is applied, as shown in FIG. 5A. When an RF pulse is interdigitated with a period of no RF, sidebands of excitation (FIG. 5B) are created at f=˜n/Δt, where Δt is the time interval between a complete cycle of RF (Δt=t_{RF}+t_{NoRF}), and n represents the nth sideband generated. This pulse construct was initially described as a DANTE pulse and used to generate frequency selectivity prior to the widespread availability of shaped RF pulses. See Freeman, R. and G. A. Morris, The ‘DANTE’ experiment. J Magn Reson, 2011. 213(2): p. 244-6; and Morris, G. A. and R. Freeman, Selective excitation in Fourier transform nuclear magnetic resonance. J. Magn. Reson., 1978. 29: p. 433-462, each of which is hereby incorporated by reference herein in its entirety.

In general, the spin response as a function of frequency, SR(ν), can be approximated from the Fourier transform, [symbol] { }, of the RF waveform via Eq. 3 in the small pulse angle limit. Furthermore, for DANTE waveforms, RF(t) can be expressed as the product of a point-by-point multiplication of a comb function C(t) and a block pulse F(t). Under these conditions, the duration in time of each “tooth” of the comb defines the duration for which the rf is applied. The Fourier transform of a point-by-point multiplication of two functions (i.e. dot product) is equal to the convolution of the individual transforms.

SR(ν)≈ [symbol] {RF(t)}= [symbol] {C(t)·F(t)}= [symbol] {C(t)}⊗ [symbol] {F(t)} Eq. 3

FIG. 6 shows exemplary RF, C and F signals in time and their corresponding Fourier transforms. Since the Fourier transform of a comb function is approximated by a comb function in the frequency domain, the overall spin response will be the convolution of a comb function with the Fourier transform of a block pulse which is approximated by a sinc function. The separation of the peaks in F{C(t)}, ΔF, is given by the inverse of the separation of the individuals pulses, ΔT (FIG. 5). This concept has been used to generate multiple bands of saturation for visualizing cardiac motion.

Improving Spin Response over the selected excitation band: Using this formalism, SR(ν) can be improved over the selected excitation bands by improving the shape of [symbol] {F(t)} by choice of a different F(t). For example if the block profile of F(t) is replaced with a truncated sinc function, i.e. a conventional single slice excitation pulse, [symbol] {F(t)} can be markedly improved. See Wu, E. X., C. W. Towe, and H. Tang, MRI cardiac tagging using a sinc-modulated RF pulse train. Magn Reson Med, 2002. 48(2): p. 389-93, which is hereby incorporated by reference herein in its entirety. Using this construction the required peak amplitude of the pulse will be increased from a conventional slice selective pulse via Δt/t_{RF}, where t_{RF} is the duration of an individual “tooth” of the comb and Δt is the separation in time between the “teeth” of the comb, i.e. the interdigitation interval (IDI).

For example fo t_{RF} t_{NoRF}, the peak amplitude is increased by 2 (i.e. amplitude of conventional sinc shown as solid line FIG. 7C), however three or more bands can be generated. Additionally, although the peak power is increased 4 fold, the average power is increased only two fold due to the reduction in RF duty cycle (i.e., Δt/t_{NoRF}). FIG. 7A shows a conventional sinc shaped excitation pulse during which the RF is applied continuously, FIG. 7B shows the sinc pulse of FIG. 7A convoluted with a comb function or DANTE pulse. FIG. 7C shows an expanded region of the pulse 7B, demonstrating the interdigitation. While the sinc pulse generates a single band of excitation (FIG. 7D), the sinc-DANTE pulse generates multiple bands of excitation, as shown in FIG. 7E. Notably the multiple bands are generated with only a doubling of the power as opposed to five times the power for the conventional MB=3 pulse (Eq. 2). Thus this approach has the potential to generate multiple excitation bands at substantially lower amplitude and power deposition.

However, as seen in FIG. 7E, the bands generated: (1) vary in amplitude and are (2) distributed throughout the entire frequency space, including both unwanted bands and potentially bands of no effective excitation. Thus, this simple approach in general cannot be used to homogeneously and selectively excite a well-defined number of slices. The variation in sideband intensity is a direct result of the significant width of the pulse t_{RF_ON} applied during the IDI (i.e., in a conventional DANTE using C(t)·F(t), C(t) is a comb function where the teeth t_{RF_ON} have significant width (e.g., ¼ on or greater). Unfortunately, dramatically decreasing pulse t_{RF_ON}, to improve the uniformity of the excitation bands results in large increases in peak RF amplitude, peak and deposited power making them undesirable for human applications.

A similar scheme was also used by Norris for simultaneous multi-slice excitation. See Norris, D. G., et al., Power Independent of Number of Slices (PINS) radiofrequency pulses for low-power simultaneous multislice excitation. Magn Reson Med, 2011. 66(5): p. 1234-40, which is hereby incorporated herein by reference in its entirety. However in Norris's method, the sinc shaped Dante pulse is not applied in a continuous selection gradient (see FIG. 2A), but rather the selection gradient is applied as a triangular waveform only during t_{NoRF} (FIG. 8A). This generates an indefinite number of relatively closely spaced excitation bands with the number controlled only by the extent of spatial coverage of the RF coils. Due to limits on gradient rise times to achieve sufficient strength during t_{NoRF}˜100 us, t_{RF}+t_{NoRF}˜200 us [5], placing the sidebands at 5 kHz intervals. Although in principle this method generates a large/indefinite number of sidebands, the small spacing of the sidebands creates artifacts in the presence of lipids and significant magnetic field inhomogeneity. In comparison, where the gradient is constant throughout the entire pulse, as shown in FIG. 8B t_{RF}+t_{NoRF} is only limited by RF waveform resolution which is ˜1-10 us, such that sidebands spaced by 50-500 kHz are possible. This reduces fat and inhomogeneity related artifacts by a factor of 10 to 100.

### SUMMARY OF THE EMBODIMENTS

In accordance with one embodiment of the invention, a method of generating Radio Frequency (RF) pulses RF(t) in a Multi-band (MB) Magnetic Resonance Imaging (MRI) system is presented. The method may include determining C(t), wherein C(t) is a dirac comb function, the period of the comb function Δt determining the frequency separation of the excitation bands, Δf, where Δf=1/Δt (therefore, for example, a C(t) may be determined that provides a predetermined and/or desired frequency separation of the excitation bands). The method may further include determining F₂(t), wherein F₂(t) is a single band frequency selective pulse, and wherein a F₂(t) may be determined so as to provide a predetermined and/or desired envelope of RF(t) during the interdigitation interval (IDI), and/or a predetermined and/or desired shape of individual excitation frequency bands in the frequency domain. The method may further include determining F₁(t) wherein an F₁(t) may be defined to provide a desired and/or predetermined shape of RF(t) during the Δt, and/or may be determined so as modulate uniformity and/or provide the number of individual excitation bands in the frequency domain. RF(t) pulses are generated in the MRI system simultaneously with an MRI gradient so as to simultaneously excite multiple slices within a subject, wherein the RF(t) pulses are of the form RF(t)=F₁(t)⊗C(t)·F₂(t), which create a spin response SR(ν)≈ [symbol] {F₁(t)}· [symbol] {C(t)}⊗ [symbol] {F₂(t)}.

In accordance with another embodiment of the invention, a system of generating Radio Frequency (RF) pulses RF(t) in a Multi-band (MB) Magnetic Resonance Imaging (MRI) system is provided. The system includes a controller configured to: determine C(t) as described above, wherein C(t) is a dirac comb function, the period of the comb function Δt determining the frequency separation of the excitation bands, Δf, where Δf=1/Δt; determine F₂(t) as described above, wherein F₂(t) is a single band frequency selective pulse, wherein F₂(t) defines the envelope of RF(t), and shape of individual excitation frequency bands in the frequency domain; and/or determine F₁(t) as described above, wherein F₁(t) defines the shape of RF(t) during the Δt, and is used to modulate uniformity and/or number of individual excitation bands in the frequency domain; and generate RF(t) pulses in the MRI system simultaneously with an MRI gradient so as to simultaneously excite multiple slices within a subject, wherein the RF(t) pulses are of the form RF(t)=F₁(t)⊗C(t) [symbol] F₂(t), which create a spin response SR(ν)≈ [symbol] {F₁(t)}· [symbol] {C(t)}⊗ [symbol] {F₂(t)}.

In accordance with yet another embodiment of the invention, a non-transitory tangible computer program product in a computer-readable medium for generating Radio Frequency (RF) pulses RF(t) in a Multi-band (MB) Magnetic Resonance Imaging (MRI) system is provided. The product includes: program code for determining C(t), as described above, wherein C(t) is a dirac comb function, the period of the comb function Δt determining the frequency separation of the excitation bands, Δf, where Δf=1/Δt; program code for determining F₂(t), as described above, wherein F₂(t) is a single band frequency selective pulse, wherein F₂(t) defines the envelope of RF(t), and shape of individual excitation frequency bands in the frequency domain; and/or program code for determining F₁(t), as described above, wherein F₁(t) defines the shape of RF(t) during each Δt, and is used to modulate uniformity and/or number of individual excitation bands in the frequency domain; and program code for generating RF(t) pulses in the MRI system simultaneously with an MRI gradient so as to simultaneously excite multiple slices within a subject, wherein the RF(t) pulses are of the form RF(t)=F₁(t)⊗C(t)·F₂(t), which create a spin response SR(ν)≈ [symbol] {F₁(t)}· [symbol] {C(t)}⊗ [symbol] {F₂(t)}.

In embodiments related to any of the above-described embodiments, the MRI system may have transmission coils and reception coils defining a RF coil field of view, wherein a target field of view in the slice direction is substantially equal to the RF coil field of view, and wherein determining F₁(t) may include defining F₁(t) as a rectangular pulse having a time in which no RF is applied, so as to provide a desired and/or predetermined variation in the spin response of the individual excitation bands and/or provide a desired and/or predetermined reduction in deposited power.

In further embodiments related to any of the above-described embodiments, the MRI system has transmission coils and reception coils defining a RF coil field of view, wherein a target field of view in the slice direction is substantially equal to the RF coil field of view, and wherein determining F₁(t) may include defining F₁(t) as a gaussian, sinusoid, hanning, or hamming, so as to provide a desired and/or predetermined variation in the spin response of the individual excitation bands and/or provide a desired and/or predetermined reduction in deposited power. Determining F₁(t) may include: adding a period where no RF is applied; and increasing amplitude of RF(t) and/or shortening duration of t_{RF}, so as to provide a desired and/or predetermined increase in the bandwidth of F₁(t).

In still further embodiments related to any of the above-described embodiments, the MRI system has transmission coils and reception coils defining a RF coil field of view, wherein a target field of view in the slice direction is less than the RF coil field of view, and wherein determining F₁(t) may include making F₁(t) frequency selective, so that sidebands of excitation outside the target field of view are removed or reduced. Determining F₁(t) may include adding a period where no RF is applied; and increasing amplitude of RF(t) and/or shortening duration of F₁(t), so as to provide a desired and/or predetermined increase in the bandwidth of F₁. F₁(t) may be one of a sinc function and a Shinnar Le Roux pulse.

### BRIEF DESCRIPTION OF THE DRAWINGS

The foregoing features of embodiments will be more readily understood by reference to the following detailed description, taken with reference to the accompanying drawings, in which:

Prior Art FIG. 1A illustrates the basic components of a typical Magnetic Resonance Imaging (MRI) system. Prior Art FIG. 1B illustrates some details in a traditional MRI resonance system, and Prior Art FIG. 1C illustrates other structural features of a traditional MRI system;

Prior Art FIGS. 2A-2C illustrate multi-band imaging. FIG. 2A shows a timing diagram. FIG. 2B illustrates multiple spatially separated slices that are excited and FIG. 2C shows the positions of the slices (red lines) on an image of a patient's head;

Prior Art FIG. 3 shows data from the simultaneously excited slices to be separated into their original slices;

Prior Art FIGS. 4A-C illustrates conventional multi-band imaging. FIG. 4A shows the RF pulse. FIG. 4B shows the excitation bands. FIG. 4C shows in more detail one of the excitation bands;

Prior Art FIGS. 5A-B shows creation of multiple excitation bands by interdigitating RF pulses with periods where no RF is applied. FIG. 5A shows interdigitating RF pulses with a period of no RF, and FIG. 5B shows the sidebands of excitation created;

Prior Art FIG. 6 shows exemplary Dante RF, C and F signals in time and their corresponding Fourier transforms;

Prior Art FIG. 7A shows a conventional sinc shaped excitation pulse during which the RF is applied continuously. Prior Art FIG. 7B shows the sinc pulse of FIG. 7A convoluted with a comb function or DANTE pulse. Prior Art FIG. 7C shows an expanded region of the pulse 7B, demonstrating the interdigitation. Prior Art FIG. 7D shows a single band of excitation based on the pulse of FIG. 7A. Prior Art FIG. 7E shows multiple bands of excitation based on the sinc-DANTE pulse of FIG. 7B;

Prior Art FIG. 8A shows a sinc shaped Dante pulse applied in a discontinuous selection gradient. Prior Art FIG. 8B shows a sinc shaped Dante pulse applied in a continuous selection gradient;

FIG. 9 shows exemplary waveforms for F₁(t), C(t), F₂(t) and RF(t), and their corresponding fourier transforms, in accordance with various embodiments of the invention;

FIG. 10A illustrates where the extent of the desired slices object or subject is to be imaged is limited by the spatial extent of the receive coils, in accordance with an embodiment of the invention. FIG. 10B illustrates where the extent of the desired slices is not constrained by the spatial extent of the receive coils (i.e., the field of view of the transmission and reception coils in the slice direction is larger than the target field of view in the slice direction), in accordance with various embodiments of the invention;

FIGS. 11A-11E show pulse construction and resulting band excitation when the Target Slice FOV is limited by the Coil, in accordance with an embodiment of the invention. FIG. 11A shows slice excitation across the skull. FIG. 11B shows the RF pulse waveform. FIG. 11C shows a 100 us portion of the RF pulse, near the center of the pulse. FIG. 11D shows calculated spin responses of the various F₁(t) pulses. FIG. 11E shows the overall homogeneity of the spin responses;

FIGS. 12A-12E show pulse construction and resulting band excitation when the Target Slice FOV is limited by the Coil, and additionally where F₁(t) is a gaussian pulse construct, in accordance with an embodiment of the invention. FIG. 12A shows slice excitation across the head. FIG. 12B shows the RF pulse waveform. FIG. 12C shows a 100 us portion of the RF pulse, near the center of the pulse. FIG. 12D shows calculated spin responses of the various F₁(t) pulses. FIG. 12E shows the overall homogeneity of the spin responses;

FIGS. 13A-13C compare the performance of an n=±4 gaussian pulse and an n=±2 gaussian with an equivalent delay, in accordance with an embodiment of the invention. FIG. 13A shows a 100 us portion of the RF pulse, near the center of the pulse. FIG. 13B shows the RF pulse waveform. FIG. 13C shows the homogeneity of the spin responses;

FIGS. 14A-14H show pulse construction and resulting band excitation when the Target Slice FOV is not limited by the RF Coil FOV (i.e., when the target slice FOV is smaller than the sensitive FOV of the coils utilized for reception or transmission), in accordance with an embodiment of the invention. FIG. 14A shows the parameters used in the design of the F₁(t) pulse in the frequency domain. FIG. 14B shows a menu used to edit variables associated with F₁(t). FIG. 14C shows F₁(t). FIG. 14D shows band excitation relative to F₁ in the frequency domain. FIG. 14F shows the RF pulse waveform. FIG. 14G shows a 100 us portion of the RF pulse, near the center of the pulse. FIGS. 14G and 14H compare the calculated response for the designed pulse and a conventional MB=3 pulse over 250 Kz and 2 kHz respectively, C(t)·F(t); and

FIG. 15 is a flow diagram for a methodology for providing Radio Frequency (RF) pulses that selectively excite a well-defined number of substantially homogeneously frequency bands in a Multi-band (MB) Magnetic Resonance Imaging (MRI) system, in accordance with an embodiment of the invention.

### DETAILED DESCRIPTION OF SPECIFIC EMBODIMENTS

In illustrative embodiments of the invention, a system and methodology for providing Radio Frequency (RF) pulses that selectively excite a well-defined number of substantially homogeneously frequency bands in a Multi-band (MB) Magnetic Resonance Imaging (MRI) system is presented. Further embodiments of the invention provide for providing multi-band excitation pulses which can advantageously reduce power deposition and or peak excitation amplitude. Details are provided below.

In accordance with various embodiments of the invention, the MRI controller (see FIG. 1) is configured to formulate and provide RF(t) pulses as a function of three elements: 1) a new driving function modulating the homogeneity/relationship of the individual bands of excitation relative to each other, F₁(t); 2) a dirac comb function C(t) in which t_{RF} is very short compared to the IDI (for example, in various embodiments, less than 1/100 Δt or less than 1/10 Δt), generating the position of the sidebands C(t); and 3) a driving function, F₂(t) for controlling the overall shape of the individual selected bands (Eq. 4). The resulting Fourier transform, F, of the RF waveform RF(t) from Eq. 4 is expressed in Eq. 5.

RF(t)=F₁(t)⊗C(t)·F₂(t) Eq. 4

SR(ν)≈F{RF(t)}=F{F₁(t)}·F{C(t)}⊗F{F₂(t)} Eq. 5

Under this construct, the overall homogeneity of the bands and number of the bands is controlled by the additional driving function F₁(t) which adds additional shaping of the RF during the intervals defining the individual periods, t_{RF}. Thus the problem reduces to defining a suitable function F₁(t) which achieves the desired response [symbol] {F₁(t)} in the frequency space.

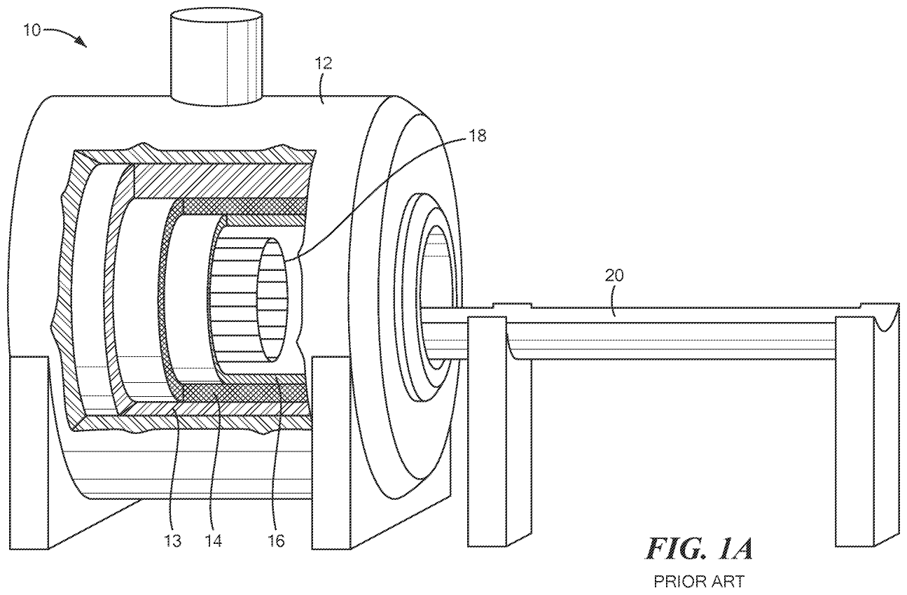

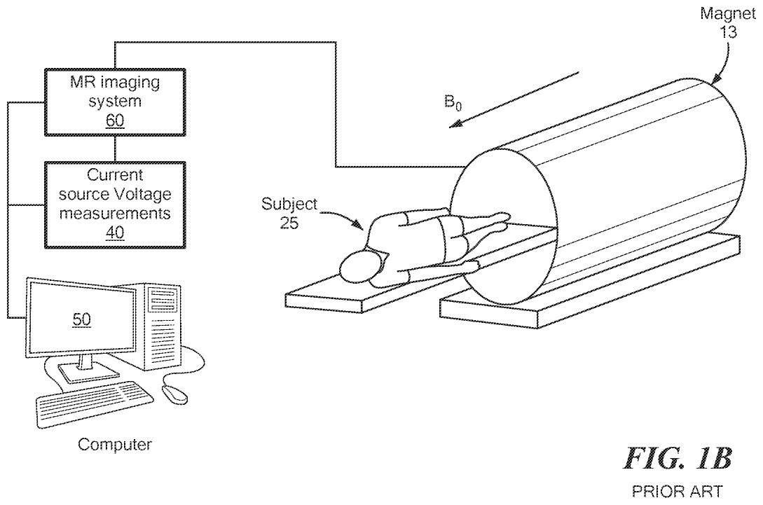

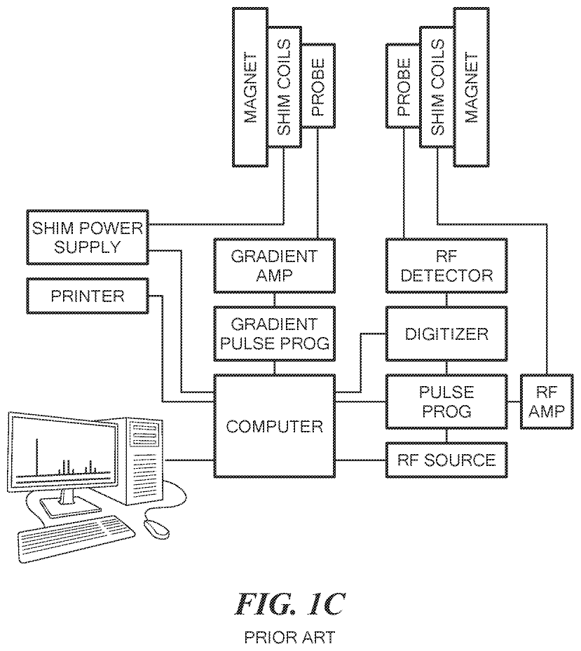

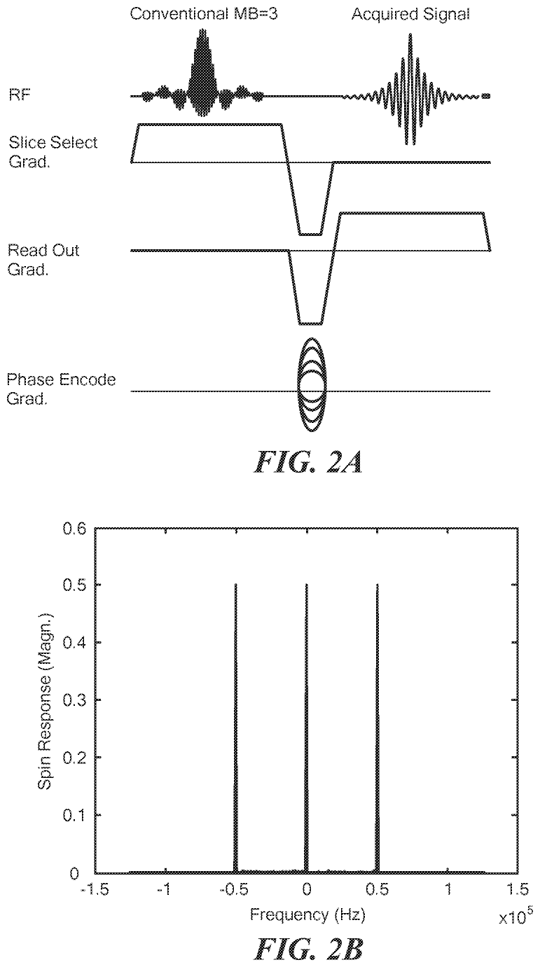

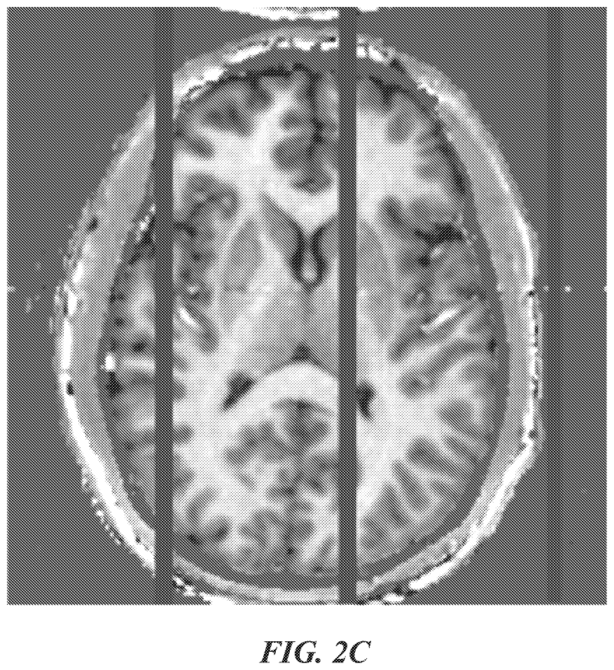

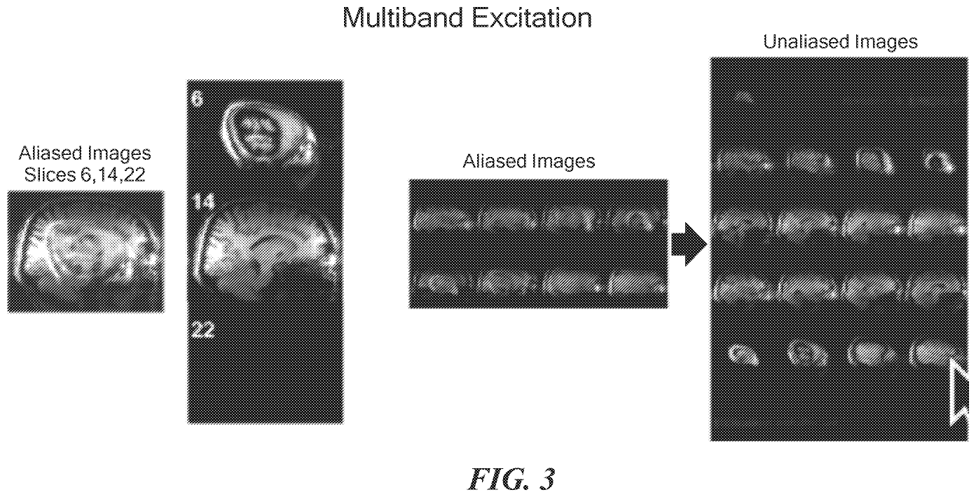

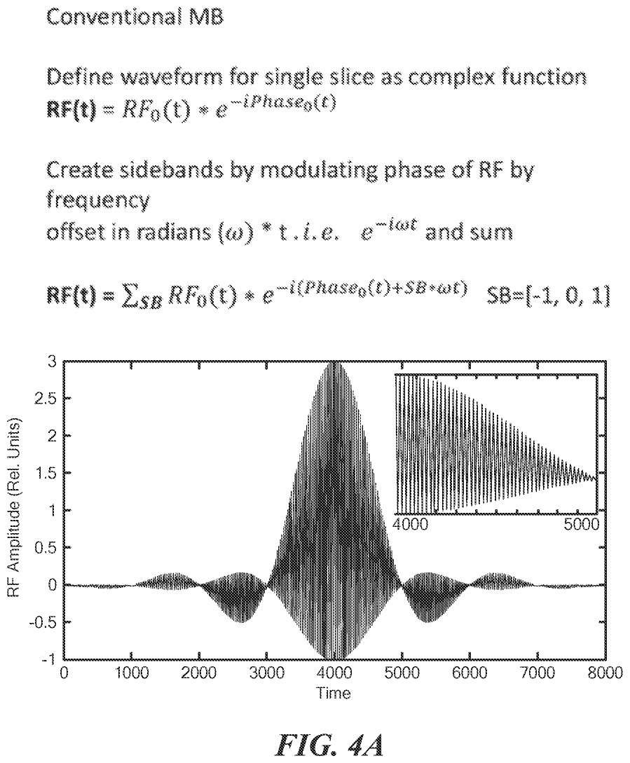

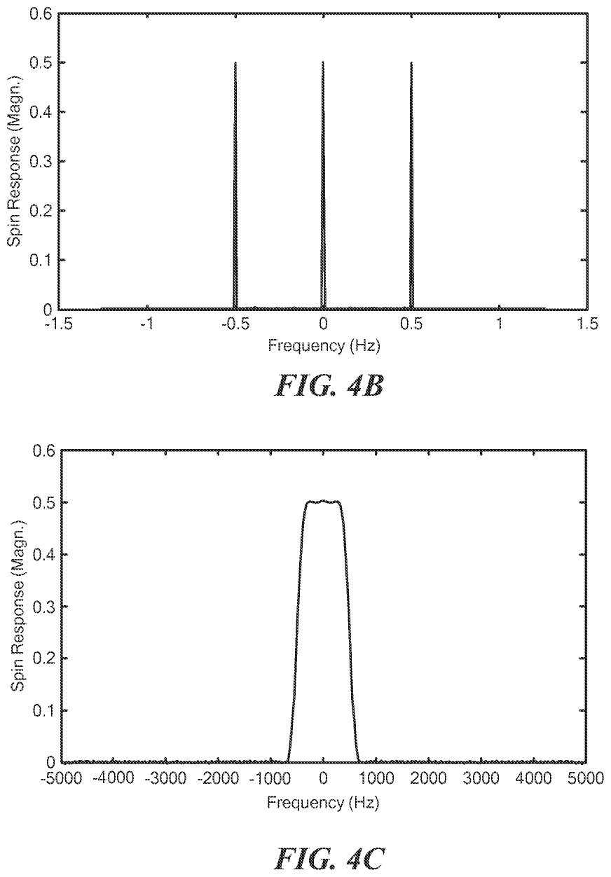

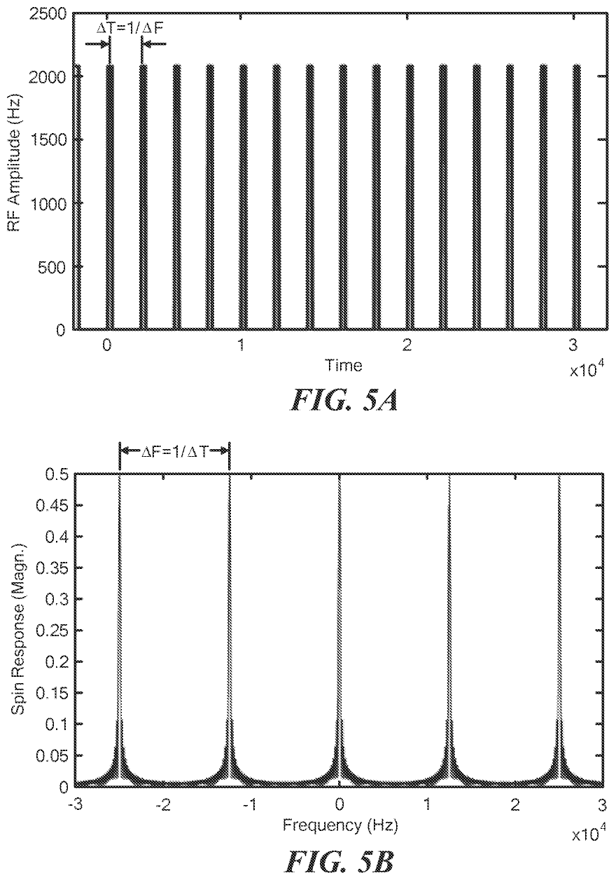

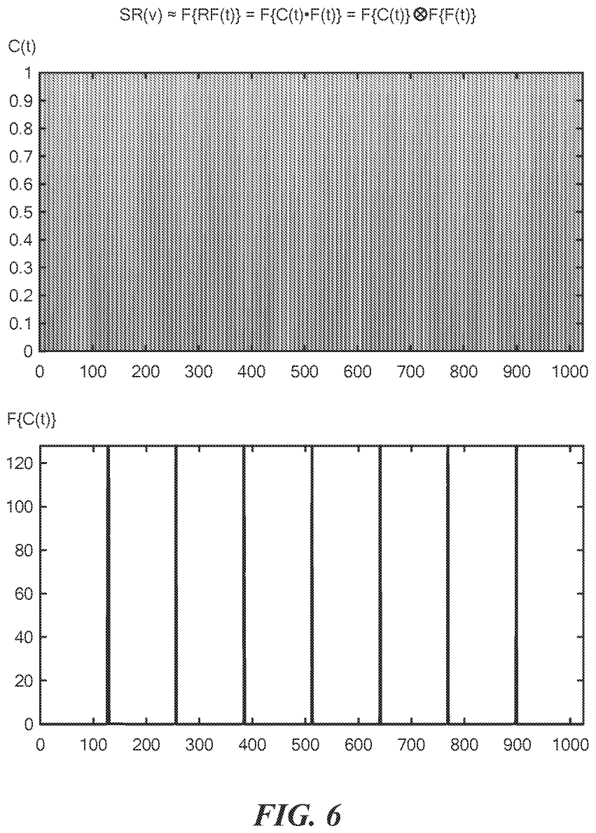

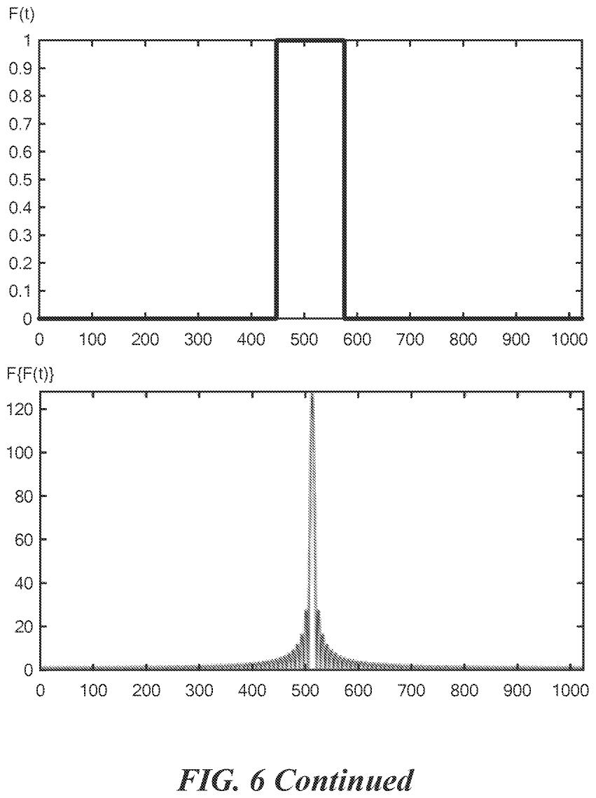

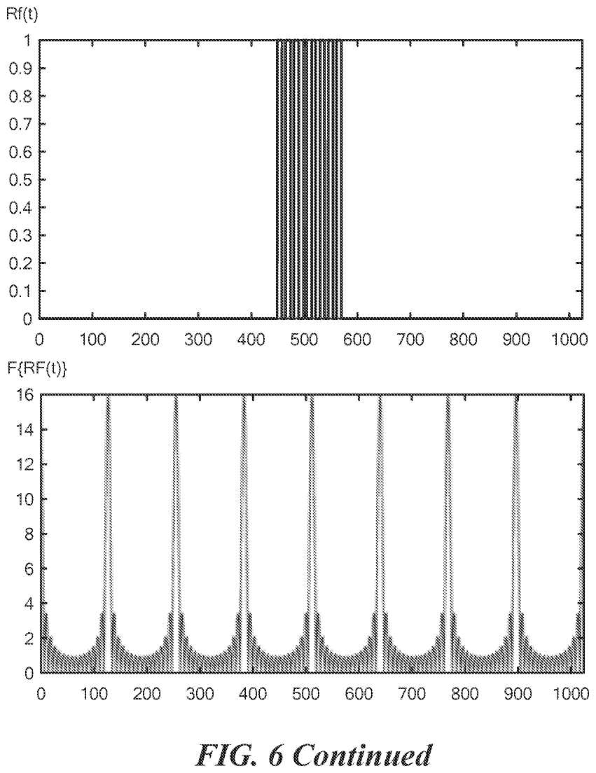

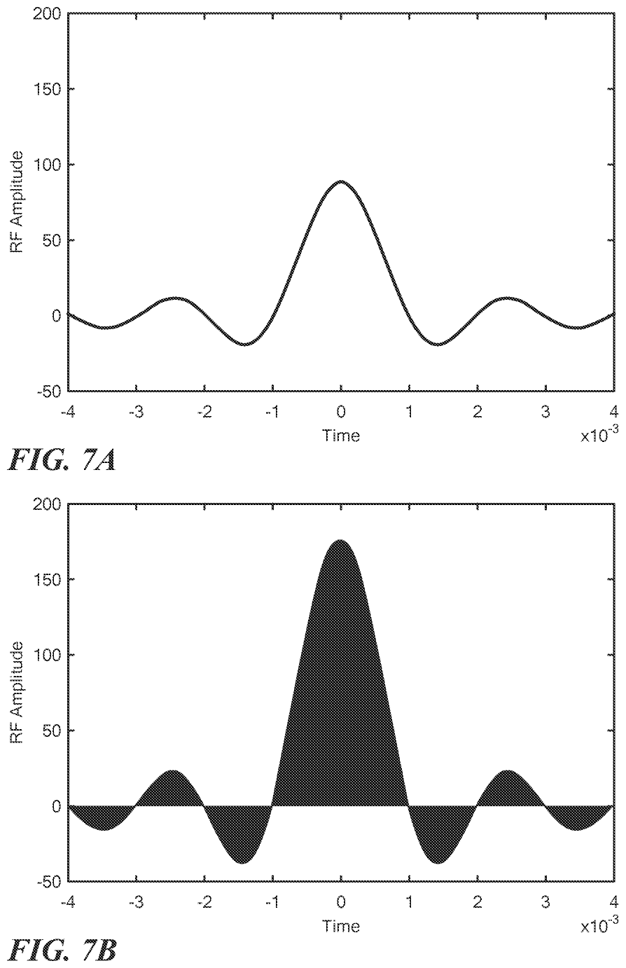

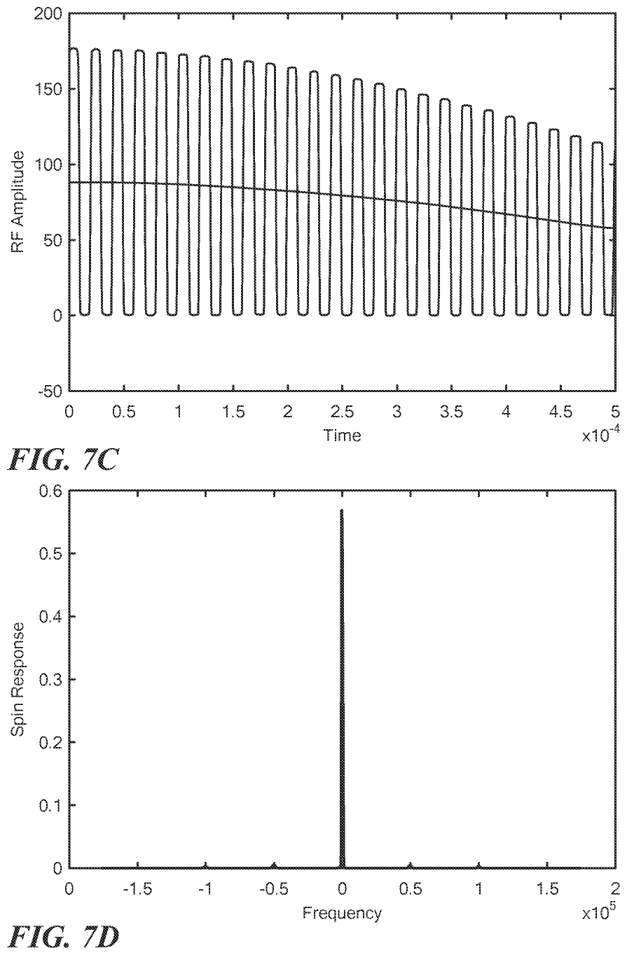

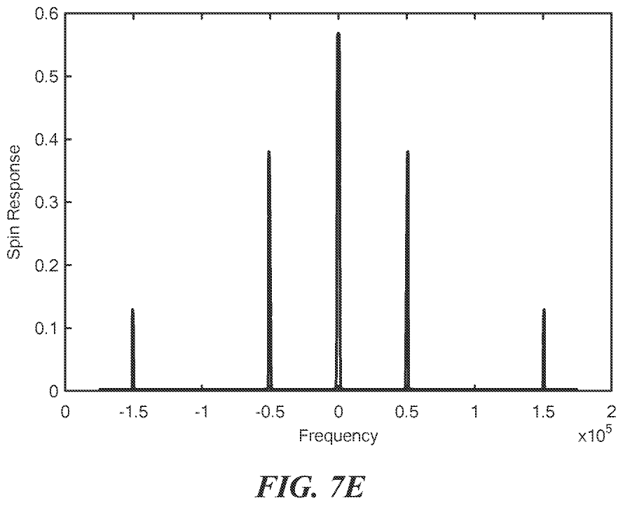

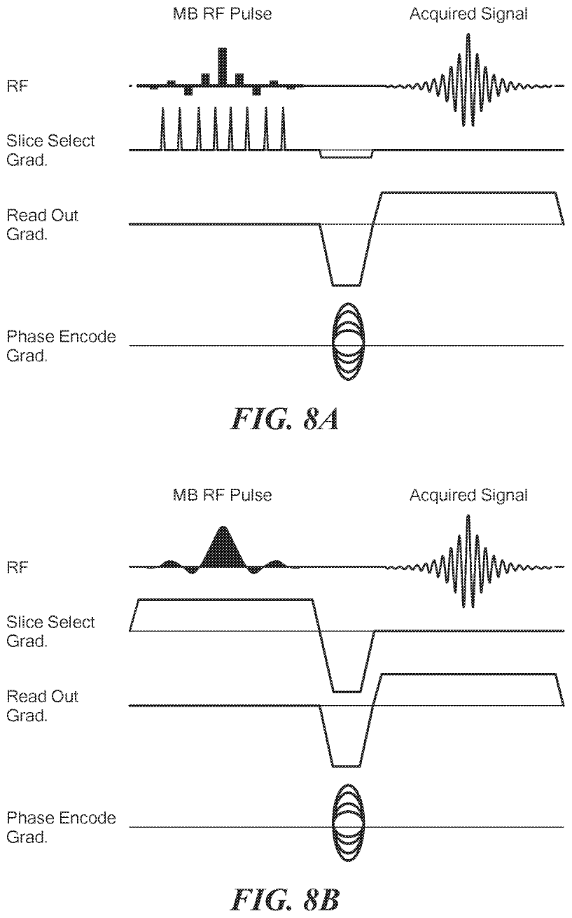

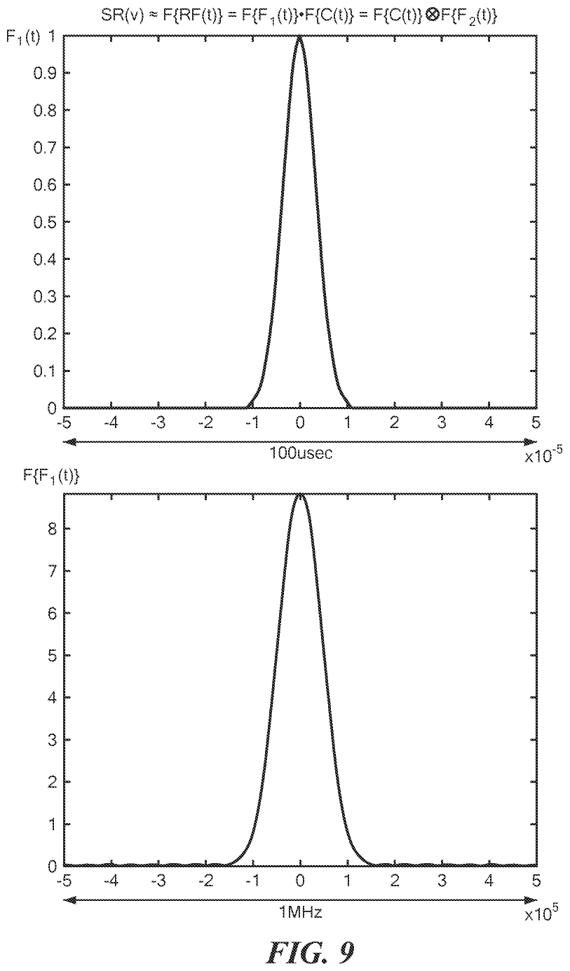

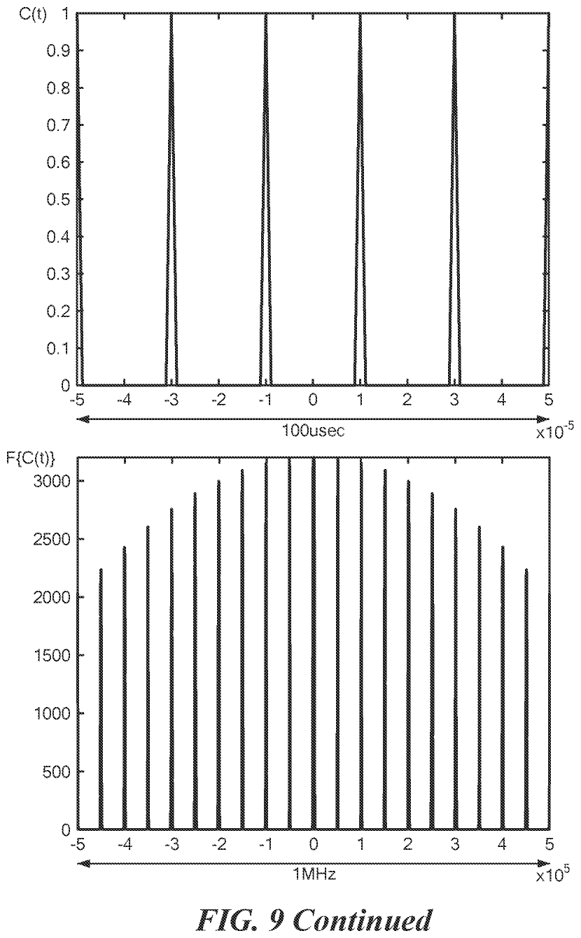

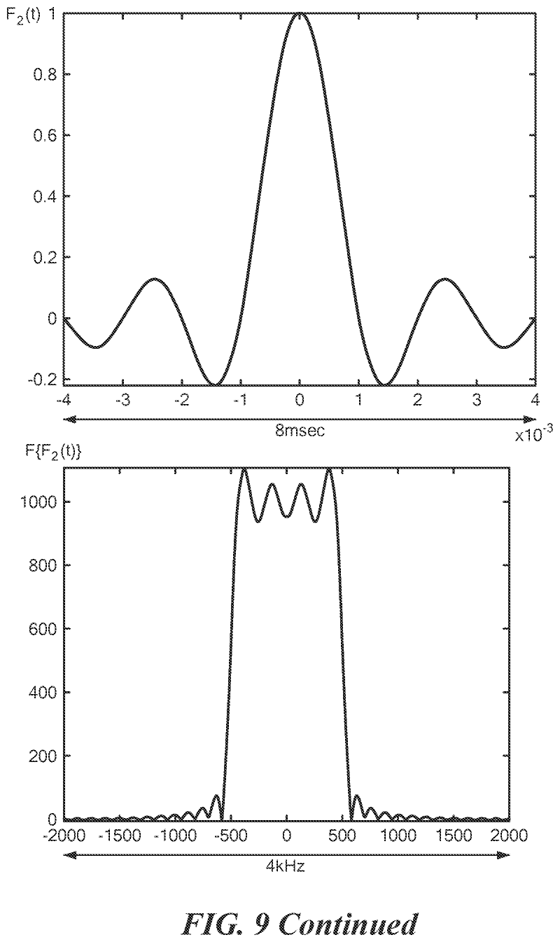

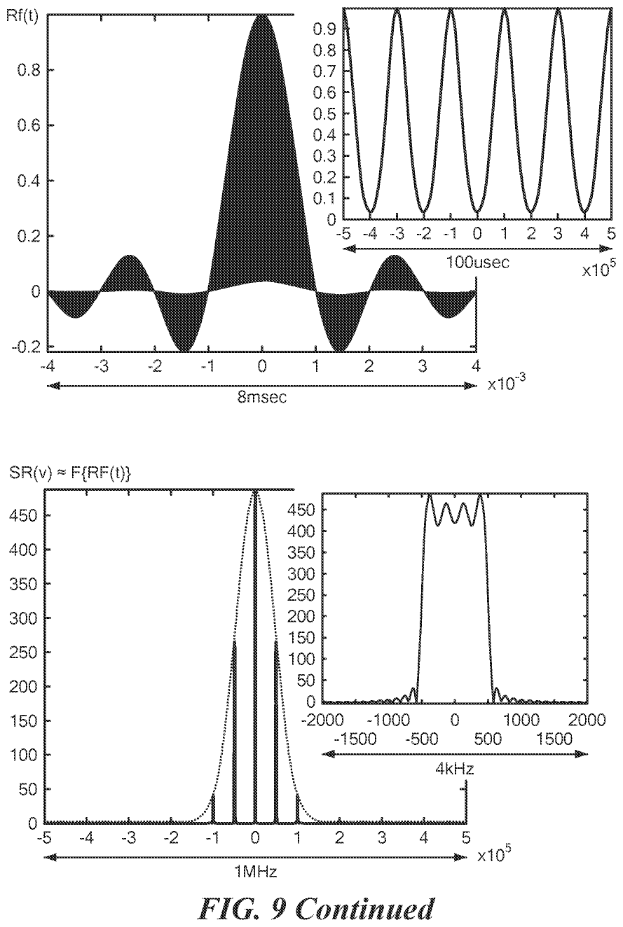

FIG. 9 shows exemplary waveforms for F₁(t), C(t), F₂(t) and RF(t), and their corresponding fourier transforms, in accordance with various embodiments of the invention. F₁(t) is a gaussian function of 20 us in length, C(t) is a dirac comb function (Δt/t_{RF}=0.05, IDI=20 us) and F₂(t) is a sinc function of 8 ms in length. Note that the relative amplitudes of the excitation bands in SR(ν) follows the distribution defined by [symbol] {F₁(t)}, i.e, the dotted lines superimposed on the plot of SR(ν). The overall shape of each excitation band follows the shape of [symbol] {F₂(t)}, i.e. the insert plot on SR(ν).

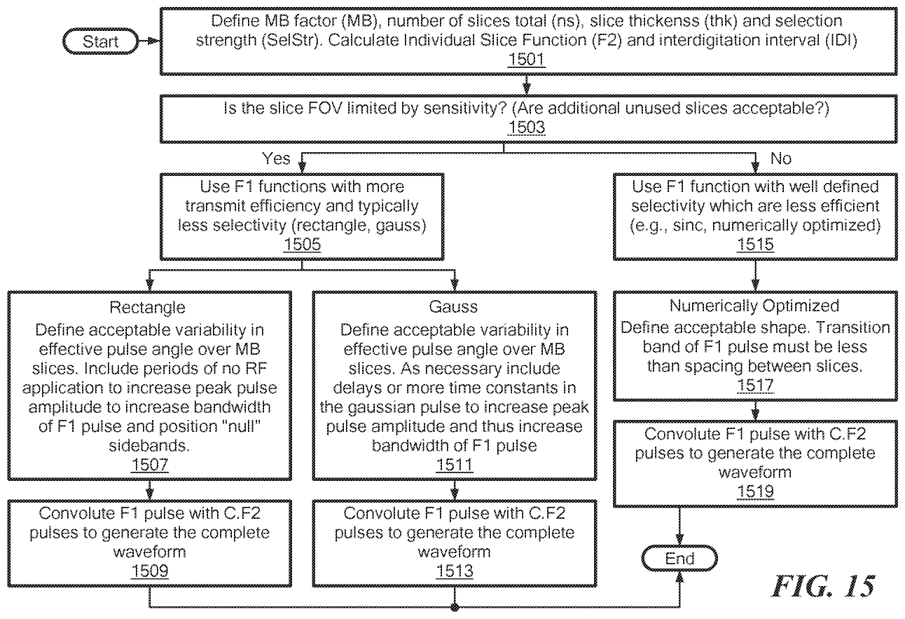

FIG. 15 is a flow diagram for a methodology for providing Radio Frequency (RF) pulses that selectively excite a well-defined number of substantially homogeneously frequency bands in a Multi-band (MB) Magnetic Resonance Imaging (MRI) system, in accordance with an embodiment of the invention.

At step 1501, various factors may initially be defined and/or determined. For example, and without limitation, MB factor (MB), number of slices total, slice thickness and selection strength (SelStr), F₂ and the interdigitation interval (IDI) may be defined and/or determined. Illustratively, to determine the IDI given Slice FOV=150 mm, MB=3, Sel. Str.=1 kHz/mm, the slice FOV/MB (mm) would equal 50 mm, the slice FOV/MB (Hz) would equal 50 kHz, and the IDI=1/Slice FOV(Hz)=0.000020.

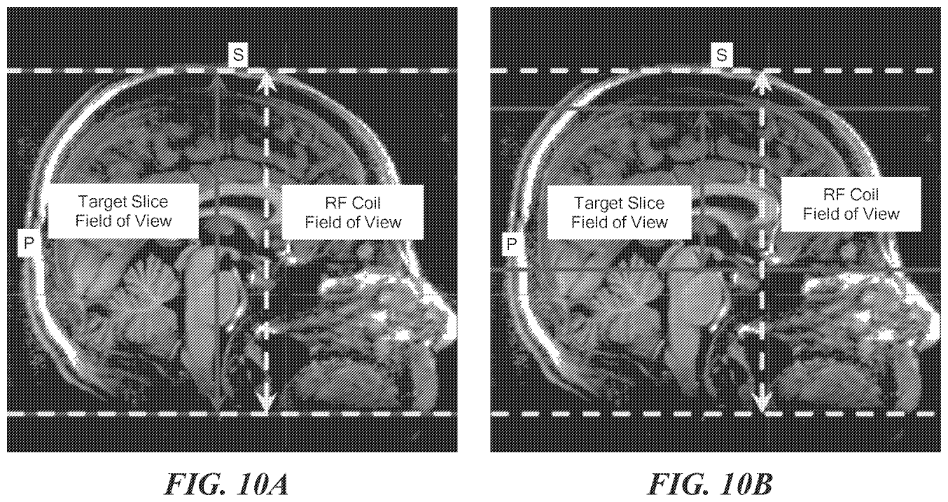

Definition of Slice FOV and Impact on Pulse Construction: Unlike the conventional MB excitation construct, (see Eq. 2) which creates a well-defined number of sidebands, the use of an interdigitated pulse generally creates many sidebands, some of which are potentially unwanted. However, it is important to recognize that the MB reconstruction process relies on the use of multiple receive coils which have differing spatial sensitivities to resolve the aliasing of imaging data. Typically the receive coils are arranged in rows (typically perpendicular to the plane of the slice) circumscribing the object, as shown in FIG. 10A. This effectively limits the spatial extent in the slice direction which contributes significant signal to be a region slightly larger than the spatial extent of the coils. Thus, if the extent of the object or subject which is to be imaged is limited by the spatial extent of the receive coils, excitation bands outside of that region will not result in significant signal and can therefore be ignored, as shown in FIG. 10A. This may significantly simplify the design of the multiband pulse and reduce issues to, with limitation, the problem of improving the homogeneity of the response. However, if the extent of the desired slices is not constrained by the receive coil sensitivity (see, for example, FIG. 10B, in which the receive coil sensitivity expands further than the target slice field of view), the presence of unwanted excitation bands may result in contamination. Thus, referring back to FIG. 15, the next step in the generation of an optimal MB pulse is to define the extent to which the desired slice FOV is limited by the receive coil array, step 1503.

Pulse Construction when the Target Slice FOV is Limited by the Coil

When the target slice FOV is limited by either the transmission coil or the receive array (i.e. target slice FOV>RF Coil field of view) considerations of unwanted sidebands are eliminated (see FIG. 10A). Under this condition, the homogeneity of SR(ν) over the individual excitation bands and/or the transmit efficiency may be of more relevance when considering F₁(t), step 1505. Thus the desired frequency response, F{F₁(t)} does not have to be limited to excite bands only over the target slice FOV, rather it may advantageously be designed to be as homogeneous as possible over the target slice FOV. This observation is important since it: 1) directly affects the needed duration of F₁(t) and 2) its overall efficiency in generating a given rotation angle. The duration of the F₁(t) pulse is limited by Δt, which we will refer to as the interdigitation interval (IDI), which is given by the inverse of the separation in Hz of the individual excitation bands. More selective pulses, i.e. pulses with “narrower” sides (transition bands) and broader tops, typically resemble truncated “sinc” functions which tend to have both positive and negative lobes (e.g. see FIG. 7A) which decreases the average amplitude over the waveform, requiring greater peak amplitude. This also increases the overall power deposition since the power is the integral of the square of the amplitude. Thus simpler shapes such as block pulses, step 1507, or gaussian pulses, step 1511, which are more efficient, may be used.

Block Pulse Constructs for F₁(t): The conventional sinc-DANTE pulse described in FIG. 7B, may be described as: an F₁(t) pulse consisting of a pulse and a delay; 2) a comb function C(t); and 3) an F₂(t) function generating a single slice selective pulse (e.g. sinc pulse), in accordance with various embodiments of the invention. The bandwidth of a block pulse (see FIG. 6A and accompanying text) is governed entirely by its amplitude/duration for a fixed low tip angle. Since the maximal duration of the pulse is limited by the IDI of the comb function and its net tip angle is constrained by F₂(t), the only “free” parameter is the ratio of t_{NoRF}/t_{RF}, with higher values associated with increased RF amplitude during t_{RF} so as to achieve the requisite pulse angle.

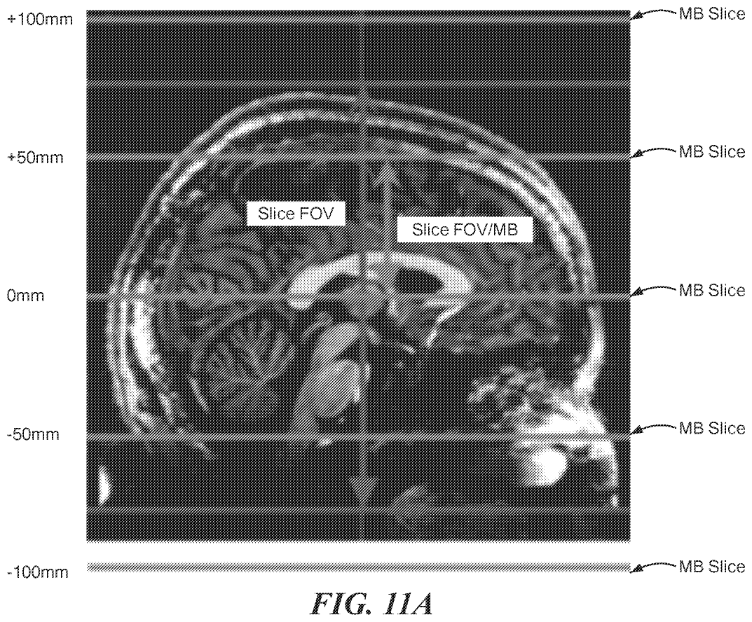

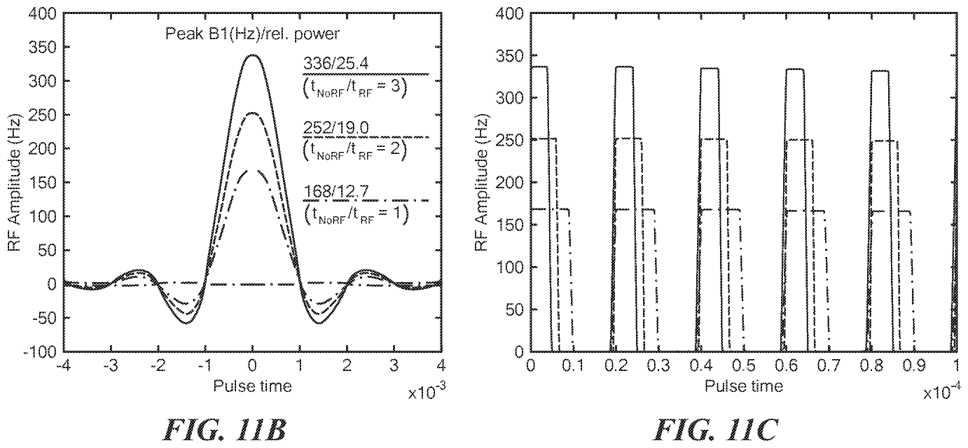

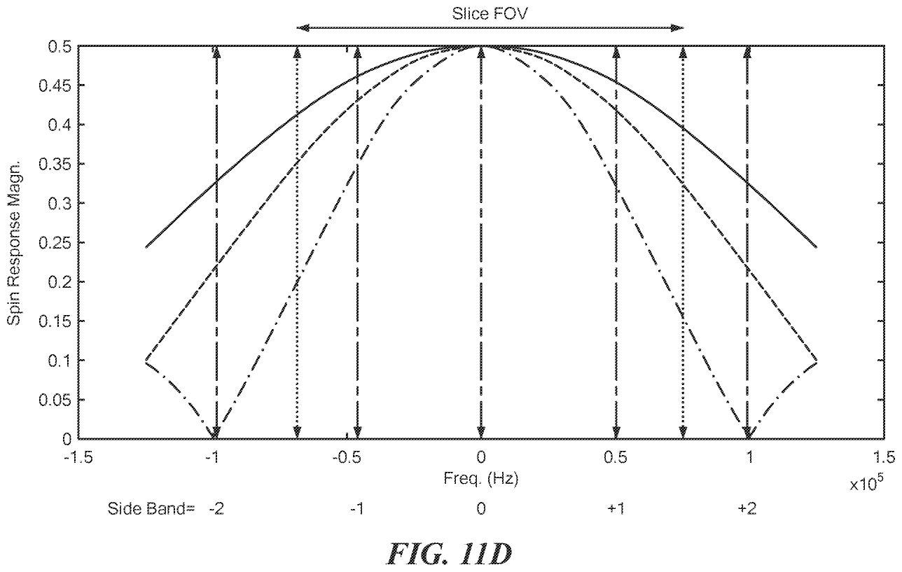

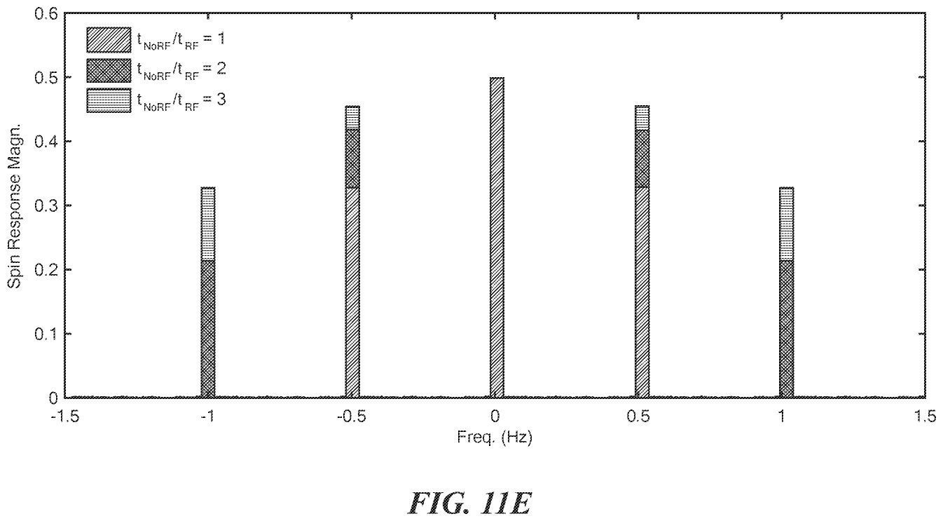

To provide a consistent comparison for the following sections, the numerical examples use realistic parameters, i.e. MB=3 (3 slices˜horizontal lines within the target slice FOV), as shown in FIG. 11A, a target slice FOV of 150 mm, a slice thickness of 1 mm, and a slice selection strength of 1 kHz/mm. This yields 150 slices, with an IDI of 20 us (see FIG. 11C) and an F₂(t) length of 8 ms for a net pulse angle of 30° (FIG. 11B). Finally the F₂(t) pulse uses a numerically optimized Shinnar-LeRoux pulse (bandwidth-time-product (BTP) of 8 (FIG. 11B) with a ripple and rejection amplitude of <0.1%. Displayed in FIGS. 11B and 11C are the resulting RF waveform and a 100 us portion near the center of the pulse, as determined by equation 4 (and referring back to FIG. 15, step 1509). Superimposed are the waveforms for cases of t_{NoRF}/t_{RF}=1, 2, 3). FIG. 11D shows the calculated spin response (e.g., using Bloch equations) for block pulses of 10, 6.67 and Sus respectively corresponding to t_{NoRF}/t_{RF}=1, 2, 3. As expected the bandwidth of the t_{NoRF}/t_{RF}=³ pulse shows a substantial increase. This reduces the variation in amplitude in the calculated spin response of the entire pulse construct, F₁(t)⊗C(t)·F₂(t), over the excited bands, improving the overall homogeneity of the response, FIG. 11E. However, this results in a corresponding increase in peak amplitude (FIG. 11B) and power.

IDI=MB/(target slice FOV*slice selection strength) Eq. 6

Duration of F₂(t)=BTP*(slice thickness/slice selection strength) Eq. 7

Peak B1 (Maximum amplitude of RF(t))≈(t_{NoRF}+t_{RF})/t_{RF} Eq. 8

Average power (Average relative power of RF(t))≈(t_{NoRF}+t_{RF})/t_{RF} Eq. 9

For comparison purposes a conventional MB=3 pulse using the same F₂(t) driving function results in a peak amplitude of 252 Hz and an average relative deposited power of 19.1 s⁻¹. Notably t_{NoRF}/t_{RF}=1 eliminates the ±2 sideband while retaining 65% of the main band and reducing the overall peak amplitude and power by 33% in comparison to a conventional MB=3 pulse. For t_{NoRF}/t_{RF}=2, the peak amplitude and RF is virtually equivalent to a conventional MB=3 pulse and the ±1 sidebands are approximately 85% of the main band, while yielding attenuated ±2 sidebands. Finally, t_{NoRF}/t_{RF}=3 yields ±1 sidebands of >90% of the main band with ±2 sidebands of ˜65% of the main band. Thus depending upon the extent to which some variation in slice amplitude is acceptable, the pulse construct can either reduce the peak amplitude or power deposition by 33% or yield MB=5 with only a 33% increase in power above a conventional MB=3 pulse (as opposed to 167% in peak amplitude for conventional MB=5 pulse).

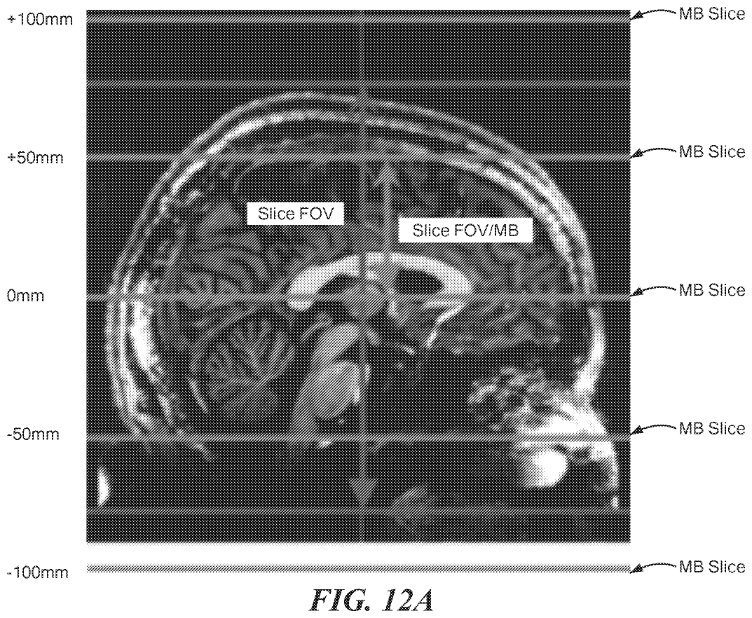

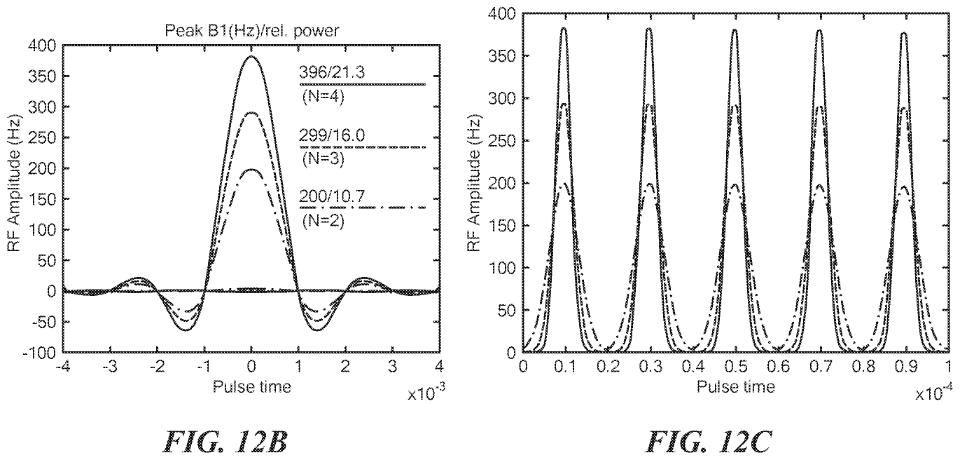

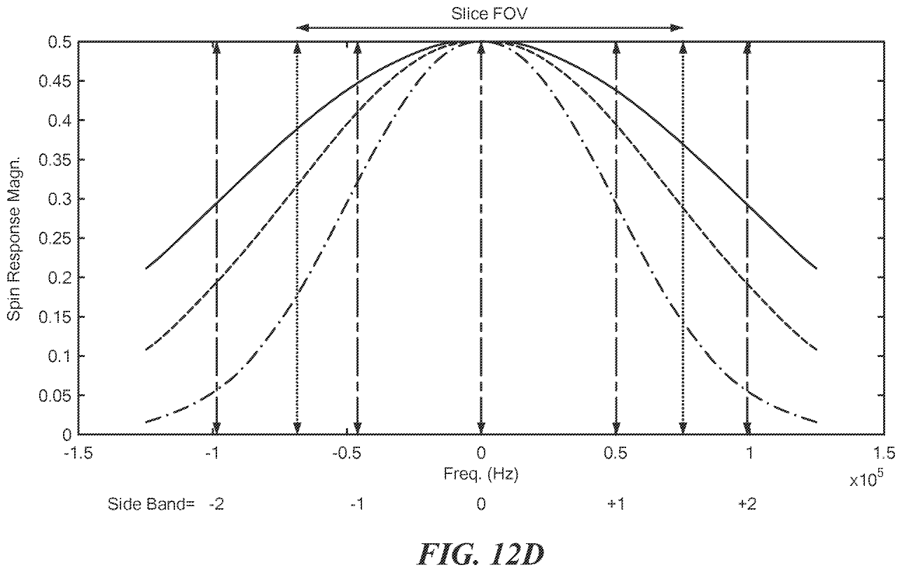

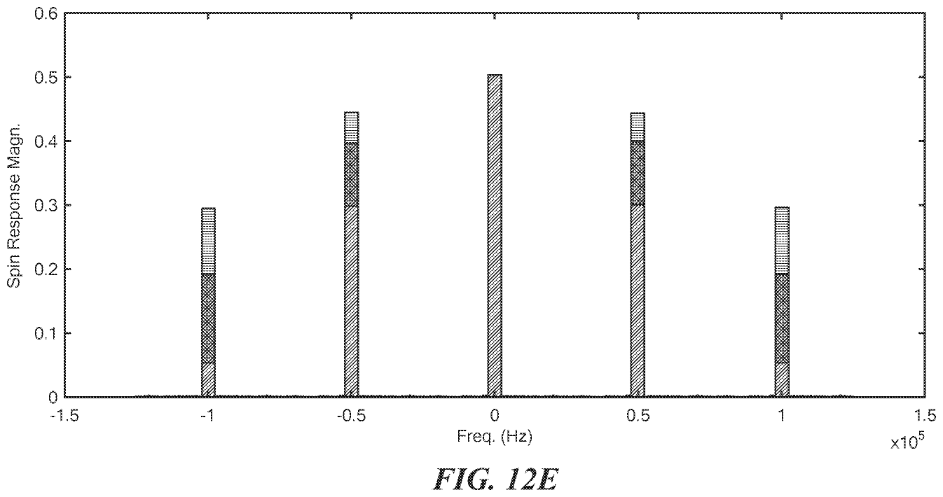

Gaussian Pulse Constructs for F(t): To increase bandwidth while attenuating the increase in power, the block pulse used for F₁(t) may be replaced with a gaussian shaped pulse. FIGS. 12A-12E show pulse construction and resulting band excitation when the Target Slice FOV is limited by the Coil, and additionally where F₁(t) is a gaussian pulse construct, in accordance with an embodiment of the invention.

For example, the Gaussian pulse may be defined by Eq. 10.

F₁(t)=e^{-t}^{²} where t=[−n, . . . ,0, . . . n] Eq. 10.

Unlike a block pulse, the RF can be applied throughout the IDI, i.e. t_{NoRF}=0. Under these conditions, the bandwidth of the pulse is modulated by the number of time constants (n) used to define the pulse as shown in FIG. 12C, with larger values of n resulting in higher peak amplitudes and greater bandwidth as shown in FIG. 12D, and correspondingly more homogeneous spin response, as shown in FIG. 12E.

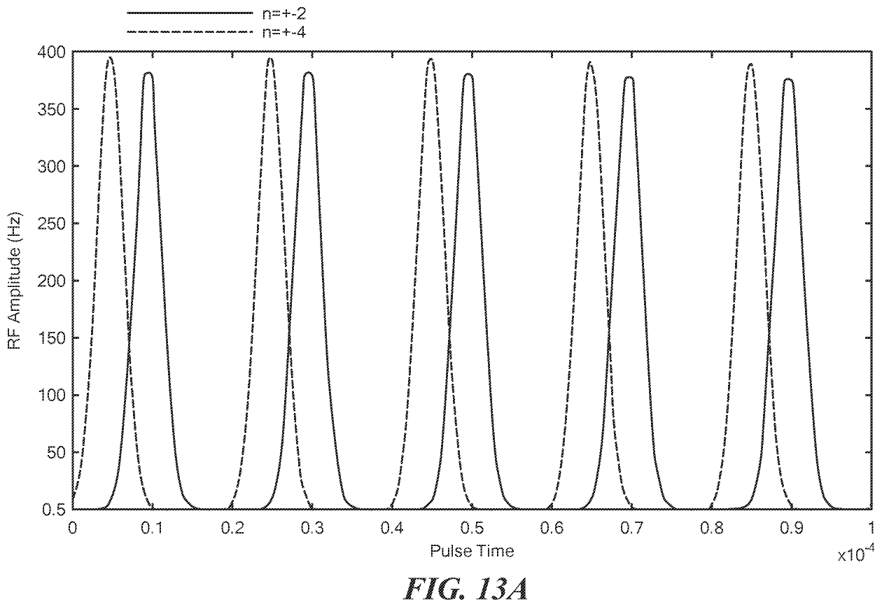

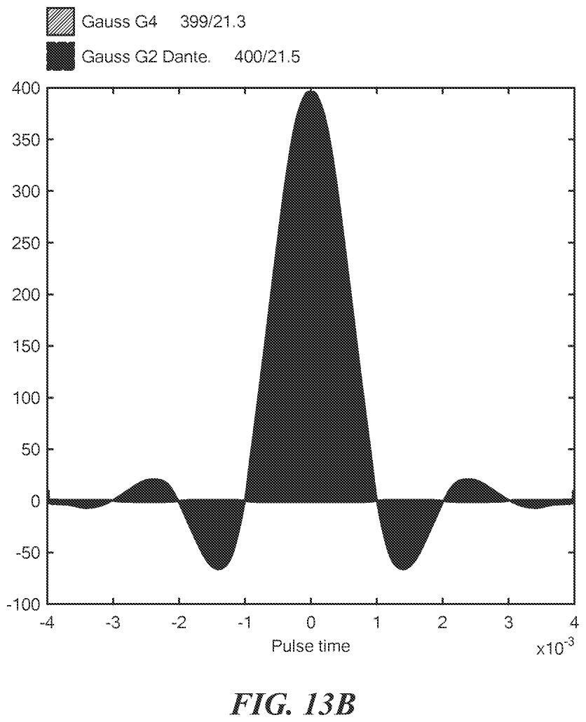

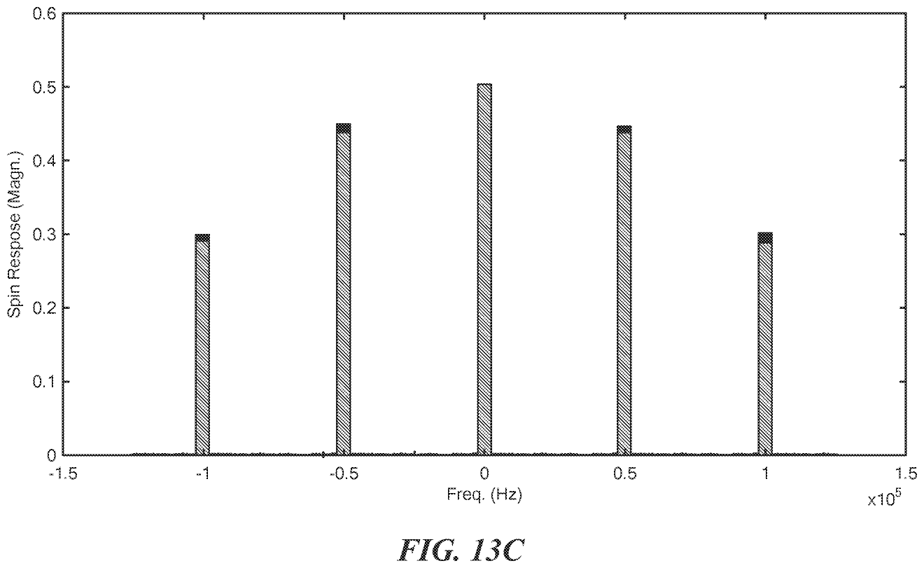

Although the peak B₁ amplitude is significantly increased relative to using a block pulse for F₁(t), the homogeneity of the sideband response is improved with only a modest increase in power deposition for n=±4 time constants. Notably n=±3 time constants yields similar performance to t_{NoRF}/t_{RF}=2, for a block F₁(t) pulse at 84% of the relative deposited power. Alternately, the performance of the n=±4 gaussian pulse, is well approximated by using an n=±2 gaussian and t_{NoRF}/t_{RF}=1. FIGS. 13A-13C compare the performance of an n=±4 gaussian pulse and an n=±2 gaussian, in accordance with an embodiment of the invention. FIG. 13A shows a 100 us portion of the RF pulse, near the center of the pulse. FIG. 13B shows the RF pulse waveform, as calculated by Equation 4 (and referring back to FIG. 15, step 1513). FIG. 13C shows the homogeneity of the spin responses. As shown in FIG. 13B, they are nearly equal in terms of power and peak RF amplitude requirements.

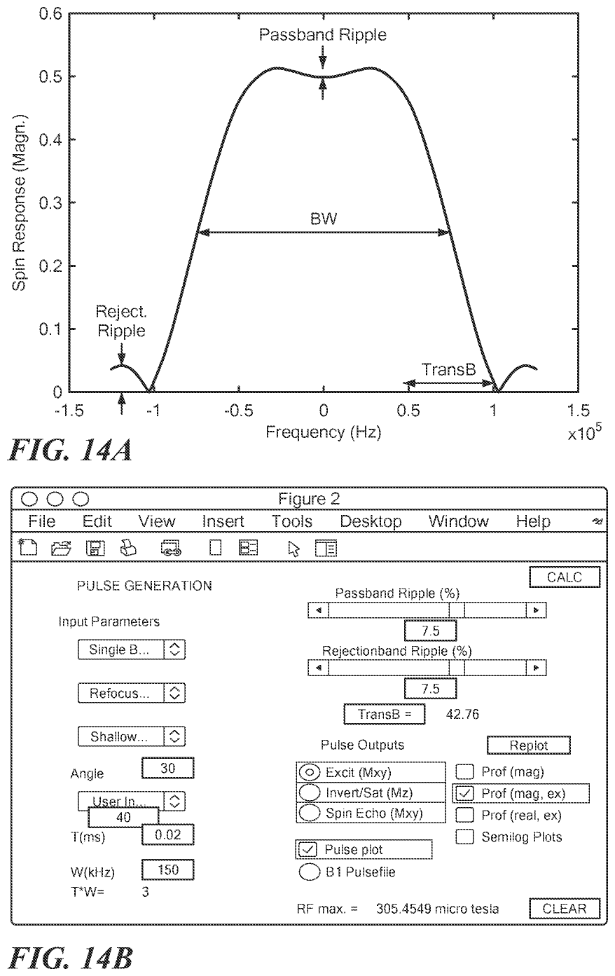

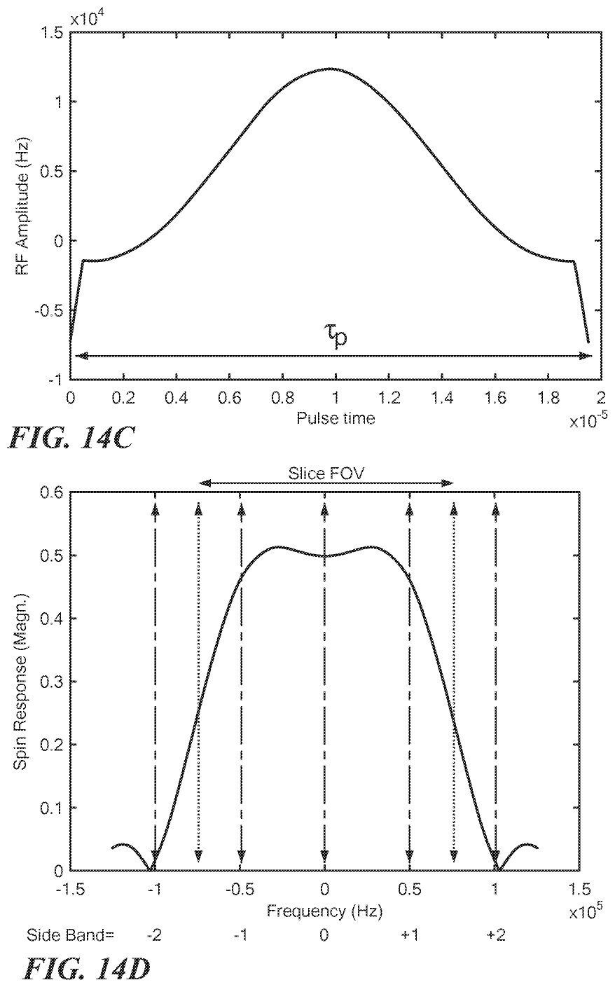

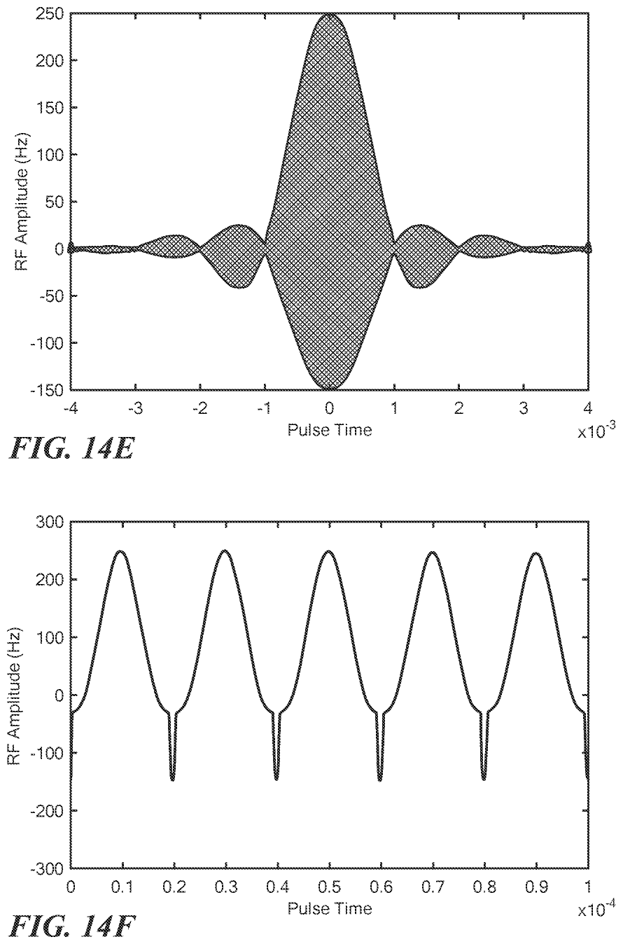

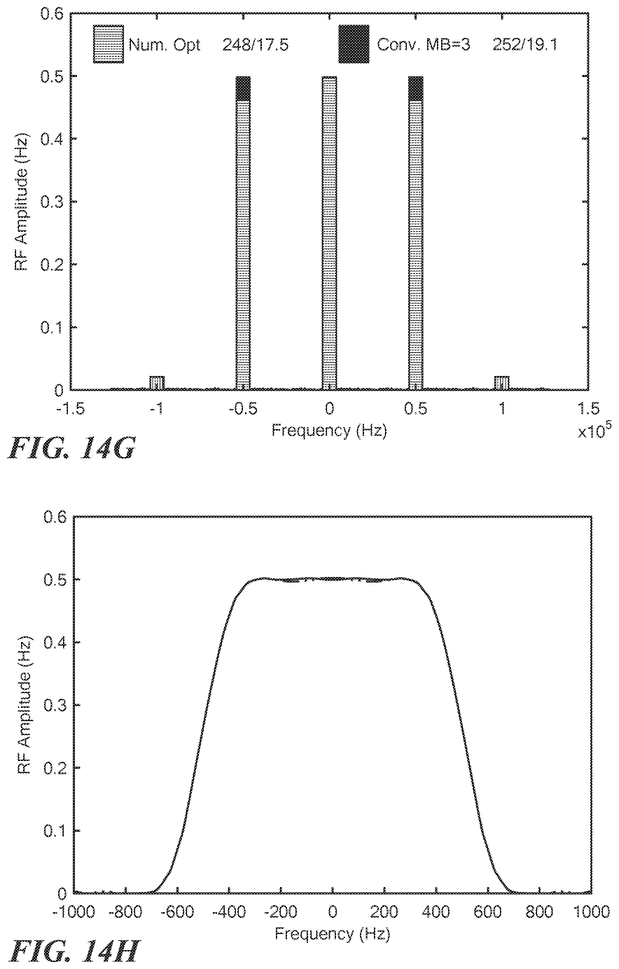

Pulse Construction when the Target Slice FOV is NOT limited by the RF Coil FOV. When the target slice FOV is smaller than the sensitive FOV of the coils utilized for reception or transmission, the number of excited bands needs to be constrained. If not, signal from outside of the target slice FOV will be acquired and contaminate the images. Under these conditions the F₁(t) pulse has to have sufficient selectivity to maintain a relatively homogeneous spin response while attenuating or eliminating all unwanted additional sidebands, step 1515 of FIG. 15. This requirement may be numerically defined/optimized, step 1517 of FIG. 15 through the following variables: 1) bandwidth (BW); 2) transition bandwidth (TransB); 3) passband ripple and 4) rejection ripple, as shown in FIG. 14A, which shows an exemplary target spin response of F₁(t) in the frequency domain, in accordance with various embodiments of the invention. To meet the first requirement that a relatively homogeneous spin response is maintained, the desired multiple excitation bands must be within the BW and the bandpass ripple should be as small as feasible. Based on a target slice FOV, 150 kHz, and IDI, 20 us, in this example, a BW-time product of 3 is used. FIG. 14B shows a menu used to edit variables associated with F₁(t) while FIG. 14C shows F₁(t). The BW-time product in turn places limits on the potential values for the transition band, pass-band ripple and rejection ripple. To meet the second requirement, the unwanted bands should fall outside of the transition bandwidth and the rejection ripple should be as sufficiently small to suppress residual unwanted sideband signals to negligible levels when factoring in both transmission and reception efficiency over those locations. Since the first unwanted sideband is likely to have the highest level of potential contamination, i.e. closest to the desired Slice FOV, placement of this sideband at or near an excitation null can be advantageous, as shown in FIG. 14D. This dictates that the transition bandwidth should be smaller than 1/IDI (FIG. 14B). Once the transition bandwidth target is set, the minimum possible ripple levels can be determined or optimized for the dictated bandwidth-time product, (FIG. 14B). FIGS. 14E (entire RF pulse) and 14F (100 us near center of pulse) display the optimized complete waveform, F₁(t)⊗C(t)·F₂(t) (see step 1519 of FIG. 15) using numerically optimized SLR pulses for both the F₁ and F₂ pulses. FIGS. 14G and 14H compare the calculated response for the designed pulse and a conventional MB=3 pulse over 250 Kz and 2 kHz respectively. Notably the optimized pulse has nearly identical spin response to the conventional MB=3 pulse with the exception of a small (<10%) reduction in the ±1 sideband amplitude and less than 5% amplitude in the ±2 sideband. The ±2 sideband can be further reduced by a systematic search varying the bandpass and rejection ripple levels to center the 1^{st} frequency null. Finally, the numerically optimized pulse shows a small decrease in both maximum peak RF amplitude, ˜1.5% and deposited relative power, 8.4%, in comparison to the conventional MB=3 pulse.

It should be noted that the spin response of the F₁ function which affects the overall performance is largely limited to the frequency positions of the sidebands. Thus substantial passband ripple is acceptable as long as the frequencies corresponding to the desired excitation bands are at a similar value. Likewise, substantial rejection ripple is also acceptable as long as the frequencies corresponding to the unwanted excitation bands are near or at nulls. This allows for the design of pulses with relatively narrow transition bands and small bandwidth-time products. Finally, similar to the gaussian F₁ pulse, the t_{NoRF}/t_{RF} may also be modified to achieve greater bandwidths in exchange for smaller bandwidth-time products.

Embodiments of the invention may be located implemented in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments also can be implemented in part as a computer program product for use with a computer system—for example, the controller of the MRI system described above. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A method of generating Radio Frequency (RF) pulses RF(t) in a Multi-band (MB) Magnetic Resonance Imaging (MRI) system, the method comprising:
determining C(t), wherein C(t) is a dirac comb function, the period of the comb function Δt determining the frequency separation of the excitation bands, Δf, where Δf=1/Δt;
determining F₂(t), wherein F₂(t) is a single band frequency selective pulse, and wherein F₂(t) defines the envelope of a pulse of RF(t), and the shape of individual excitation frequency bands in the frequency domain;
determining F₁(t) wherein F₁(t) defines the shape of RF(t) during the Δt, and is used to modulate uniformity and/or number of individual excitation bands in the frequency domain; and
generating RF(t) pulses in the MRI system simultaneously with an MRI gradient so as to simultaneously excite multiple slices within a subject, wherein the RF(t) pulses are of the form RF(t)=F₁(t)⊗C(t)·F₂(t), which create a spin response SR(ν)≈ [symbol] {F₁(t)} [symbol] {C(t)}⊗ [symbol] {F₂(t)}.

2. The method according to claim 1, further comprising producing MRI images of the subject as a function of the excitation.

3. The method according to claim 1, wherein the MRI system has transmission coils and reception coils defining a RF coil field of view, wherein a target field of view in the slice direction is substantially equal to the RF coil field of view, and wherein determining F₁(t) includes defining F₁(t) as a rectangular pulse having a time in which no RF is applied, whereby variation in the spin response of the individual excitation bands and/or deposited power is reduced.

4. The method according to claim 1, wherein the MRI system has transmission coils and reception coils defining a RF coil field of view, wherein a target field of view in the slice direction is substantially equal to the RF coil field of view, and wherein determining F₁(t) includes defining F₁(t) as a gaussian, sinusoid, hanning, or hamming, whereby variation in the spin response of the individual excitation bands and/or deposited power is reduced.

5. The method according to claim 4, wherein determining F₁(t) includes: adding a period where no RF is applied; and increasing amplitude of RF(t) and/or shortening duration of t_{RF}, such that the bandwidth of F₁(t) is increased.

6. The method of claim 1, wherein the MRI system has transmission coils and reception coils defining a RF coil field of view, wherein a target field of view in the slice direction is less than the RF coil field of view, and wherein determining F₁(t) includes making F₁(t) frequency selective, so that sidebands of excitation outside the target field of view are removed or reduced.

7. The method according to claim 6, wherein determining F₁(t) includes: adding a period where no RF is applied; and increasing amplitude of RF(t) and/or shortening duration of F₁(t), such that the bandwidth of F₁(t) is increased.

8. The method according to claim 6, wherein F₁(t) is one of a sinc function and a Shinnar Le Roux pulse.

9. A system of generating Radio Frequency (RF) pulses RF(t) in a Multi-band (MB) Magnetic Resonance Imaging (MRI) system, the system comprising:
a controller configured to:
determine C(t), wherein C(t) is a dirac comb function, the period of the comb function Δt determining the frequency separation of the excitation bands, Δf, where Δf=1/Δt;
determine F₂(t), wherein F₂(t) is a single band frequency selective pulse, and wherein F₂(t) defines the envelope of RF(t), and shape of individual excitation frequency bands in the frequency domain;
determine F₁(t) wherein F₁(t) defines the shape of RF(t) during the Δt, and is used to modulate uniformity and or number of individual excitation bands in the frequency domain; and
generate RF(t) pulses in the MRI system simultaneously with an MRI gradient so as to simultaneously excite multiple slices within a subject, wherein the RF(t) pulses are of the form RF(t)=F₁(t)⊗C(t)·F₂(t), which create a spin response SR(ν)≈ [symbol] {F₁(t)}· [symbol] {C(t)}⊗ [symbol] {F₂(t)}.

10. The system according to claim 9, wherein the controller is further configured to produce MRI images of the subject as a function of the excitation.

11. The system according to claim 9, wherein the system includes transmission coils and reception coils defining a RF coil field of view, wherein a target field of view in the slice direction is substantially equal to the RF coil field of view, and wherein the controller configured to determine F₁(t) is further configured to define F₁(t) as a rectangular pulse having a time in which no RF is applied, whereby variation in the spin response of the individual excitation bands and/or deposited power is reduced.

12. The system according to claim 9, wherein the MRI system has transmission coils and reception coils defining a RF coil field of view, wherein a target field of view in the slice direction is substantially equal to the RF coil field of view, and wherein the controller configured to determine F₁(t) is further configured to define F₁(t) as a gaussian, sinusoid, hanning, or hamming, whereby variation in the spin response of the individual excitation bands and/or deposited power is reduced.

13. The system according to claim 12, wherein the controller configured to determine F₁(t) is further configured to: add a period where no RF is applied; and increase amplitude of RF(t) and/or shorten duration of t_{RF}, such that the bandwidth of F₁(t) is increased.

14. The system according to claim 9, wherein the MRI system has transmission coils and reception coils defining a RF coil field of view, wherein a target field of view in the slice direction is less than the RF coil field of view, and wherein the controller configured to determine F₁(t) is further configured to make F₁(t) frequency selective, so that sidebands of excitation outside the target field of view are removed or reduced.

15. The system according to claim 14, wherein the controller configured to determine F₁(t) is further configured to add a period where no RF is applied; and increase of RF(t) and/or shortening duration of t_{RF}, such that the bandwidth of F₁(t) is increased.

16. The method according to claim 14, wherein F₁(t) is one of a sinc function and a Shinnar Le Roux pulse.

17. A non-transitory tangible computer program product in a computer-readable medium for generating Radio Frequency (RF) pulses RF(t) in a Multi-band (MB) Magnetic Resonance Imaging (MRI) system, the product comprising:
program code for determining C(t), wherein C(t) is a dirac comb function, the period of the comb function Δt determining the frequency separation of the excitation bands, Δf, where Δf=1/Δt;
program code for determining F₂(t), wherein F₂(t) is a single band frequency selective pulse, and wherein F₂(t) defines the envelope of RF(t), and shape of individual excitation frequency bands in the frequency domain;
program code for determining F₁(t) wherein F₁(t) defines the shape of RF(t) during the Δt, and is used to modulate uniformity and/or number of individual excitation bands in the frequency domain; and
program code for generating RF(t) pulses in the MRI system simultaneously with an MRI gradient so as to simultaneously excite multiple slices within a subject, wherein the RF(t) pulses are of the form RF(t)=F₁(t)⊗C(t)·F₂(t), which create a spin response SR(ν)≈ [symbol] {F₁(t)}· [symbol] {C(t)}⊗ [symbol] {F₂(t)}.

18. The product according to claim 17, wherein the MRI system has transmission coils and reception coils defining a RF coil field of view, wherein a target field of view in the slice direction is substantially equal to the RF coil field of view, and wherein program code for determining F₁(t) includes program code for defining F₁(t) as a rectangular pulse having a time in which no RF is applied, whereby variation in the spin response of the individual excitation bands and/or deposited power is reduced.

19. The product according to claim 17, wherein the MRI system has transmission coils and reception coils defining a RF coil field of view, wherein a target field of view in the slice direction is substantially equal to the RF coil field of view, and wherein the program code for determining F₁(t) includes program code for defining F₁(t) as a gaussian, sinusoid, hanning, or hamming, whereby variation in the spin response of the individual excitation bands and/or deposited power is reduced.

20. The product according to claim 19, wherein the program code for determining F₁(t) includes: program code for adding a period where no RF is applied; and program code for increasing amplitude of RF(t) and/or shortening duration of t_{RF}, such that the bandwidth of F₁(t) is increased.

21. The product according to claim 17, wherein the MRI system has transmission coils and reception coils defining a RF coil field of view, wherein a target field of view in the slice direction is less than the RF coil field of view, and wherein the program code for determining F₁(t) includes program code for making F₁(t) frequency selective, so that sidebands of excitation outside the target field of view are removed or reduced.

22. The product according to claim 21, wherein the program code for determining F₁(t) includes: program code for adding a period where no RF is applied; and program code for increasing amplitude of RF(t) and/or shortening duration of t_{RF}, such that the bandwidth of F₁(t) is increased.

23. The product according to claim 21, wherein F₁(t) is one of a sinc function and a Shinnar Le Roux pulse.
